(19) United States
(12) Patent Application Publication
(10) Pub. No.: US 2003/0231283 A1
(43) Pub. Date: Dec. 18, 2003

(54) Integrated test pupil device for corneal topography

(75) Inventor: Sergio Mura, Florence (IT)

Correspondence Address: LACKENBACH SIEGEL, ONE CHASE ROAD, SCARSDALE, NY, 10583, US

(73) Assignee: C.S.O. SRL, Badia a Settimo (IT)

(21) Appl. No.: 10/458,307

(22) Filed: Jun. 10, 2003

(30) Foreign Application Priority Data
Jun. 14, 2002 (IT) 02425386.6

(51) Int. Cl. A61B 3/00

(52) U.S. Cl. 351/200

(57) ABSTRACT

A test pupil device for Corneal Topography, having a videocamera in sequence with an optical group including lenses, an outside Placido disk, a fixing LED, an infrared fixing LED, a neon glow lamp and including at least one beam splitter plate. An infrared LED may be placed laterally onto the Placido disk or away from the Placido disk and is provided so to provide for a grazing light for the pupil test. The lateral LED and the fixing LED are infrared LED's so as not to interfere with the light sources to be used for the cornea test that uses frequencies in the visible light range.

### BACKGROUND OF THE INVENTION

This invention is concerned with Corneal topography and an integrated test pupil device. More specifically, the test pupil device comprises a video camera in sequence with an optical group including lenses and a Placido disk.

1. Description of Prior Art

It is known that a test of a cornea or of a pupil is basically in the field of ophthalmology. In fact the acquisited or acquired data for this test are particularly important both from a diagnostic point of view in order to have extremely precise data before performing a refractive surgery operation.

According to the prior art shown in FIG. 1, the cornea examination is carried out by a device known as a corneal topography device.

The known device as illustrated in FIG. 1 comprises a videocamera 1 for taking out an image or receiving an image coming from or transmitted through lens 2 and lens 3. This device also includes a fixing LED (light-emitting diode) 4 proximate to camera 1 and a glass semi-silver plated plate 5 so that the light from the LED 4 is reflected onto a silver-plated wall of the glass semi-silver-plated plate 5.

One or more neon glow lamps 6 is provided which during its ignition will light up the Placido disk or keratography 7 fixed by means of fasteners 8 to frame 9 which forms part of the corneal topography device. Outwardly of frame 9 and to the rear of videocamera 1 and on a front portion of the frame, a small monitor 10 is placed proximate to and connected to videocamera 1 by means of connection wire 11 to permit a prompt visualization of what is visualized or visualisated.

Wire 11 which is for the acquisition of acquired data is then connected with a second monitor and with a conventional computer, not shown.

During the functioning and operation of the corneal topography device, the patient's eye generally designated 12 is placed in an examining position and wire 13 is connected to LED 4 and wire 14 is connected to the semi-silver-plated plate 5 to provide for the electrical connections of neon glow lamps 6.

During the operation of the prior art corneal topography device 20, plate 5 is lit up by LED's 4 and reflects on its outside part what the patient or more specifically what the patient's eye observes, and that is the fixing centre.

In this situation and the operation of the prior art device 20, there is formed onto the cornea an image that is known as the first Purchinjè image, which is a digital image. This Purchinjè image, which is taken to the optic of the system, is then filmed by the videocamera 1, and the videocamera also sends the same digital image to the small monitor 10 by means of wire 11.

The digital images are then sent to a computer with a system software which permits the elaboration of the digital images.

### BRIEF SUMMARY OF THE INVENTION

The invented device is an improvement over the aforenoted prior art device 20 and in comparison provides for actuation of and obtaining the images with a normal solution in use between the bigger or larger part of the manufacturers and provides for or permits actuation with only one device for the pupil test as well.

It is of particular and great importance, in that the device of the present invention is able to point out and determine the actual pupil centre on the light alteration, which as understood is changeable from patient to patient.

Another feature of particular importance is that the same device is able to determine without any image interruption, the pupil morphology. This pupil morphology data is also changeable and peculiar to each patient.

Practically the device according to the invention is able to film pupil variations with different light conditions, and it is able to elaborate the area of the same pupil, and it is able to set for each light condition the pupil centre for each examined eye.

Accordingly, on this basis the same device can contemporaneously provide both the fixing centre and the pupil centre.

The acquisition of these points are distinct and separate, and it is of fundamental importance for a refractive surgery operation which is generally treated with a laser.

It is known that in these operations, an eximer laser is generally used. An eximer laser instrument is extremely precise so that there is the ability to avail oneself of two data sources. The ability to avail oneself of two data sources clearly provides for better operation results in comparison with those obtained when considering for these purposes the operation only of the fixing point and not knowing, in this situation, in such a continuous operation, the area variability of the pupil and consequently the precise centre of the pupil.

In fact, the pupil areas change from a maximum value, obtained without light, to a minimum value obtained with the highest quantity of light that the lamp presents inside the device. Clearly always when considering the keratometric axis, the patient will always observe the fixing point.

Obviously, by means of the videocamera filming, it is possible to know and ascertain all of the intermediate positions that are memorized so as to enable use for any computer elaboration.

### BRIEF DESCRIPTION OF THE DRAWINGS

FIG. 1 is a transverse sectional view of the corneal topography device of the prior art.

FIG. 2 is a perspective view of the corneal topography device according to the invention, together with related parts of an operational machine.

FIG.3 is a longitudinal sectional view of one embodiment of the invention passing through the center of the Placido disk 19 as illustrated in FIG. 2.

FIG. 4 is a transverse sectional view taken along section line A-A of FIG. 3.

FIG. 5 is a longitudinal sectional view of another embodiment of the invention, similar to FIG. 3 and illustrating in particular LED 10a.

FIG. 6 is a longitudinal sectional view of another embodiment according to the invention similar to FIG. 3 and illustrating in particular LED 10b; and

FIG. 7 is a longitudinal sectional view of another embodiment according to the inventions, similar to FIG. 3 and illustrating in particular LED 10c.

### DETAILED DESCRIPTION OF THE INVENTION

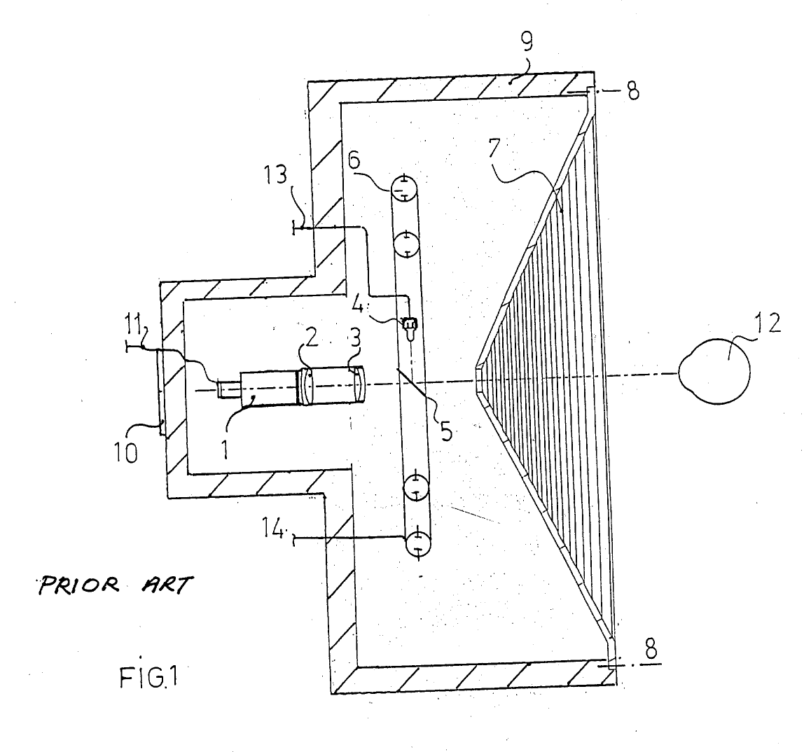

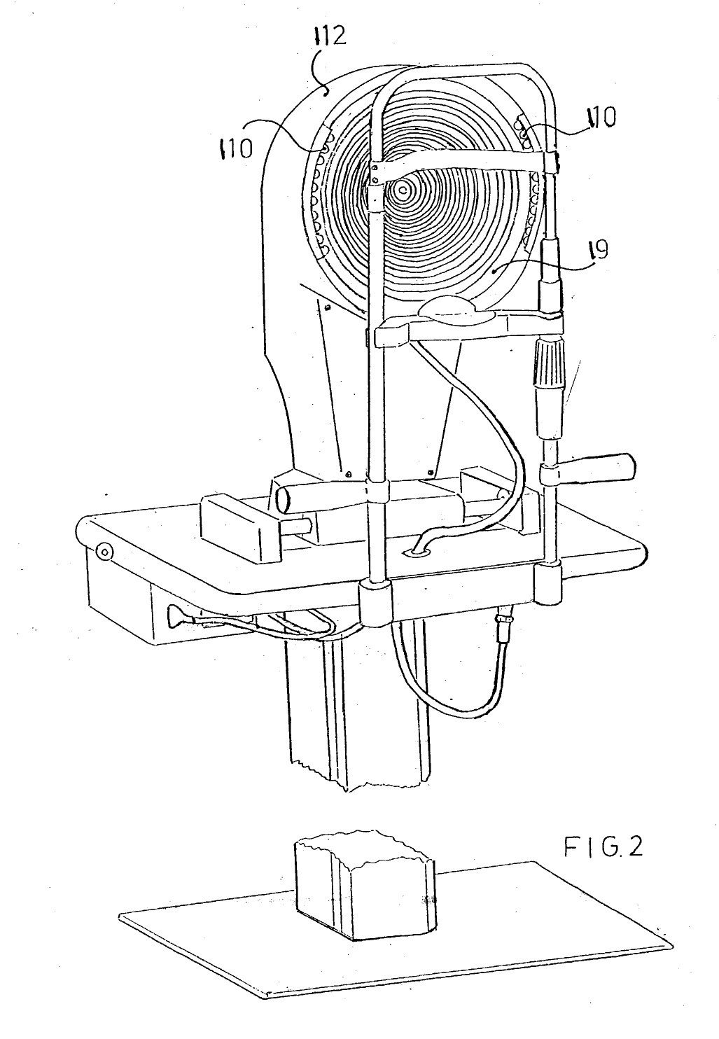

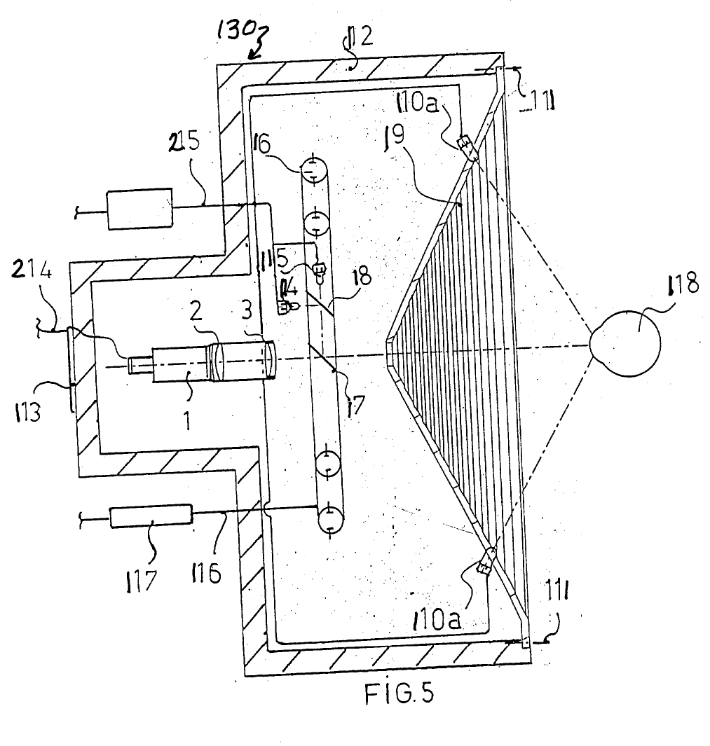

Referring now more particularly to the accompanying drawings, and in particular to FIGS. 2 to 7 in which like reference numerals designate the same parts in the different embodiments of FIGS. 2 to 7. In addition, some of the parts of the invention in common with the prior art in FIG. 1 are accorded the same reference numeral, and in other instances different numerals were used in FIGS. 2 to 7 to relate the parts to the present invention for ease of explanation.

The corneal topography device 20, according to the present invention comprises videocamera 1 as in the prior art in sequence and aligned with the optical group including lens 2 and lens 3.

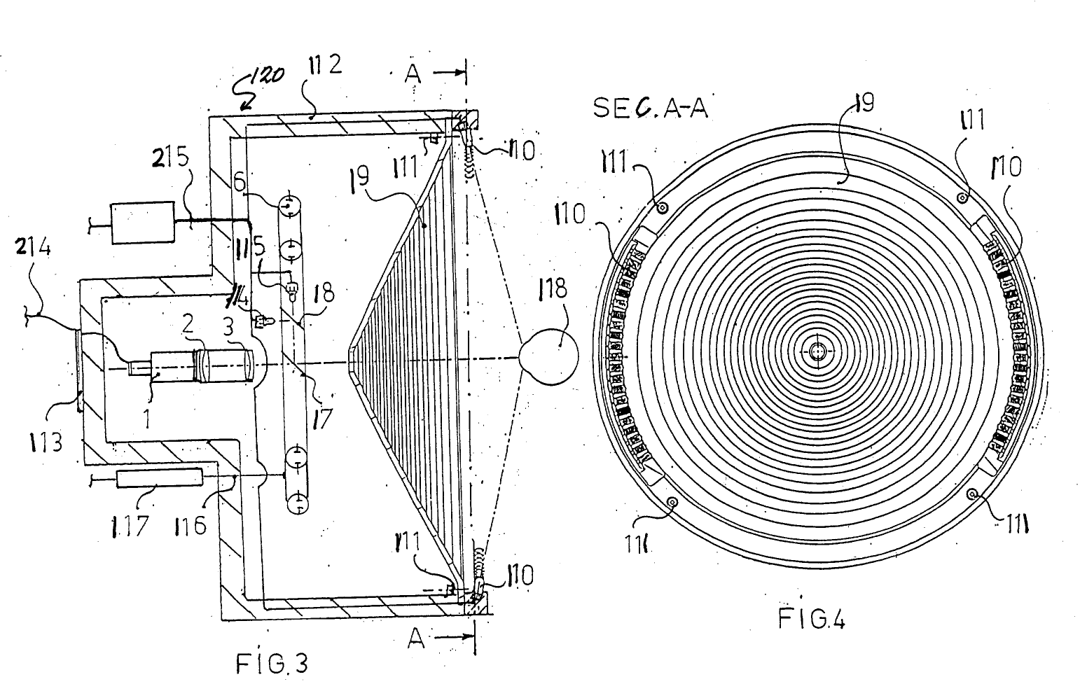

Then in accordance with a first embodiment as shown in FIG. 3, according to the invention, a fixing LED 114 and an infrared fixing LED 115 with a neon glow lamp 6 with two beam splitter plates 17 and 18 are provided proximate to the neon glow lamp 6.

A conventional Placido disk or keratograhy 19 is provided having on the outside of the corneal topography device 120 two laterally positioned infrared LED's 110. The LED's 110 are placed in this position so as to be able to provide a grazing light for the pupil test. It is noted that the Placido disk 19 is outside of lens or remote from the rays transmitted through lenses 2 and 3 to camera 1. LED's 110 and fixing LED 115 are both infrared so as not to interfere with the light sources which are used for the cornea test.

In fact, for this test, frequencies in the visible light field are used. The Placido disk 19 is mounted, by means of bolts 111, to the frame or body 112 of device 120 which serves as a housing, and the bolts 111 form a semi-arcuate configuration around the outer portion of Placido disk 119.

On the outside of housing 112, there is provided small monitor 113 in FIGS. 2 to 7, and this monitor is similar to device monitor 10 in FIG. 1, and the monitor 113 and is connected to the videocamera 1 by wire 214, in FIGS. 2 to 7.

Wire 214 also provides for and permits the sending of the signal from camera 1 to a second outside monitor connected with a computer provided with a system software able to elaborate the images.

The various LED's are connected with each other and by wires 215 and provide a network connection. Also, the neon glow lamps 6 are connected by wire 116 to a potentiometer 117 to provide for the variability of the luminous intensity.

During the use of the topography according to the invention, the patient's eye 118 to be tested is therefore contemporaneously or simultaneously subjected to the cornea test and to the pupil test.

Within the art of the invented topography device the infrared LED's, designated in FIG. 3 can 110 also to be located or provided in different positions on the basis of the use and necessity.

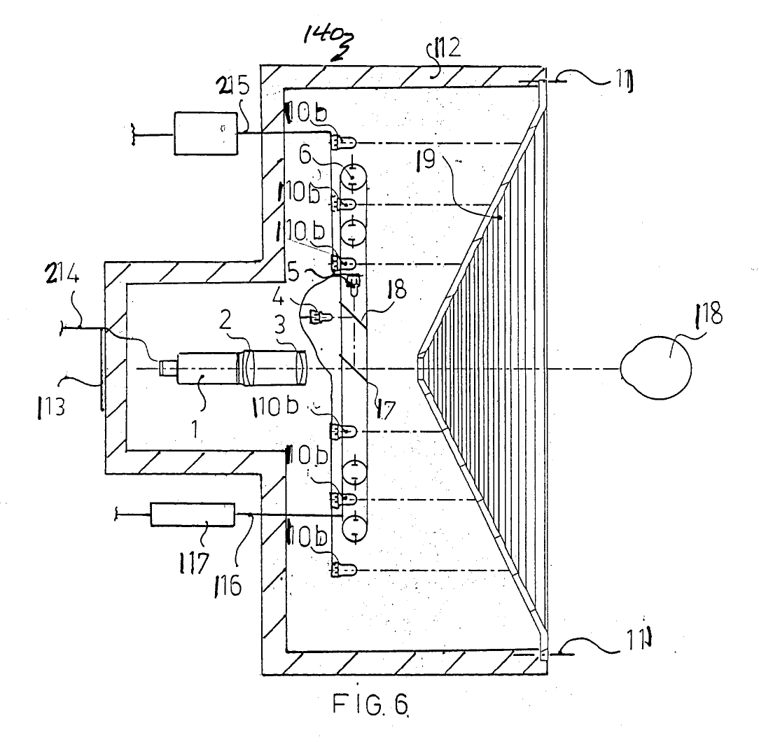

FIG. 5 shows a second embodiment in which infrared LED's 110a are placed inside the device 130 on the Placido disk 19. The LED's 110a are spaced peripherally along the Placido disk #19 and are spaced from the center or axial center of the disk and proximate but spaced from the inner walls of the housing or frame 112.

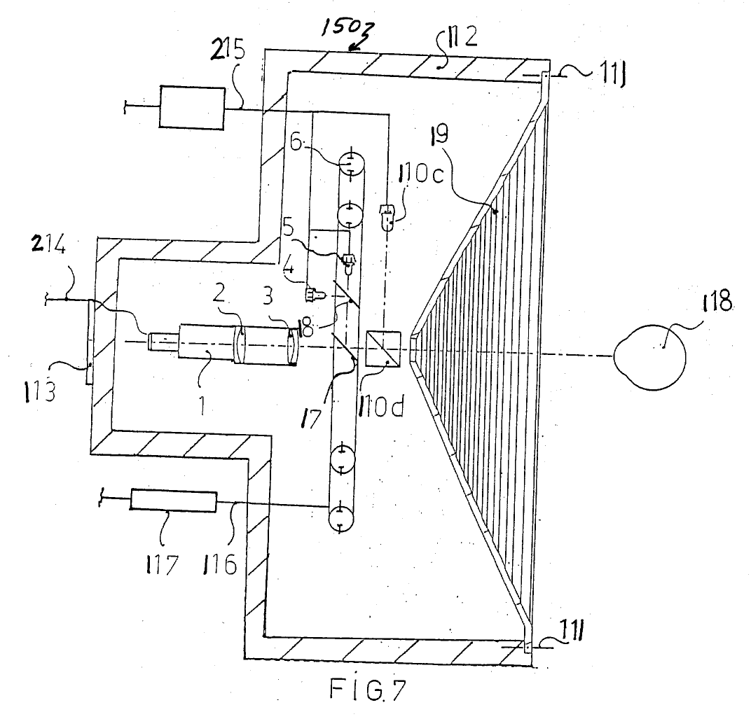

Referring now more particularly to FIG. 6 which shows a third embodiment of the device designated 140, a set of infrared LED's 110b are provided in a parallel position in comparison with or relative to glow lamp 6. These LED's 110b are placed between the semi-silver plated glass or wall 5 and lens 3, on the side of the silver plated wall 5 remote from Placido disk 118.

In a fourth embodiment the device is designated 150, as best seen in FIG. 7, and only one infrared LED 110c is provided together with a beam splitter plate 110d having the same function as the beam splitter plate 18 previous application.

LED 110c is positioned between semi-silver plated plate 5 and the Placido disk.

The invented device is illustrated in the different embodiments and in an indicative way is not limited to the details or specifics as shown in the various different embodiments illustrated FIGS. 2-7 of the drawings.

While there has been shown what is considered to be the preferred embodiments of the invention, it will be obvious to those skilled in the art that various changes and modifications may be made therein without departing from the scope of the invention.

1. video camera

2. lens

3. lens

4. LED

5. semi-silver-plated wall/glass

6. new glow lamps

7. Placido disk or Kerotography—fixed

8. fasteners

9. frame

10. Monitor

10. FIG. 3

10a. LED FIG. 5

10b. LED FIG. 6

10c LED FIG. 7

11. connection wire

12. eye—FIG. 2

13. electrical wire

14. fixing LED

15. infrared fixing LED

16. wire

17. beam splitter plate

18. beam splitter plate

20 corneal prior art device

110 LED

111. Bolts

112. device frame or body

113. monitor

114. wire

115. wires

116. wires

117. potentiometer

120. Corneal topography device

130. 2^{nd} embodying 10a is on inside of device

1. Integrated test pupil device for Corneal topography comprising:
a housing;
a videocamera in sequence with an optical group including a lens and a Placido disk located within said housing;
a fixing LED and an infrared fixing LED provided with a neon glow lamp having at least one beam splitter plate; and
at least one infrared LED associated with said Placido disk for providing grazing light for a pupil test;
said infrared LED associated with said Placido disk and said fixing LED (5) being infrared so not to interfere with light sources used for a cornea test that uses frequencies in the visible light field.

2. In the device as claimed in claim 1, including subjecting a patient's eye being tested to contemporary neotuity to the cornea test and to the pupil test.

3. In the device as claimed in claim 1, wherein the infrared LED is placeable in different positions based on use or necessity.

4. In the device as claimed in claim 1, wherein the infrared LED associated with said Placido disk is placed inside the device on the Placido disk.

5. In the device as claimed in claim 1, including providing a set of infrared LED's associated with the Placido disk in a parallel position relative to said neon glow lamp (6).

6. In the device as claimed in claim 1, wherein only one of said infrared LED associated with said Placido disk is provided with a beam splitter plate.

7. In the device as claimed in claim 1, including a camera to film the pupil centre to the light variation and to provide contemporaneously a fixing centre and a pupil centre, said LED's associated with said Placido disk being in a parallel position relative to the neon glow lamp.

8. In the device as claimed in claim 1, wherein the infrared LED is placed laterally onto said Placido disk.

9. The device as claimed in claim 1, including two beam splitter plates

10. An integrated test pupil device for corneal topography, comprising a composite arrangement including LED means relative to a Placido disk, and direction means to direct part of the bean to a monitor and part to a camera.

11. The device as claimed in claim 10, wherein said LED means includes a set of LED's in parallel with said neon glow lamp.

12. The device as claimed in claim 10, where said LED means includes LED's inside said Placido disk and said direction means comprises a beam splitter.

13. The device as claimed in claim 10, wherein said LED means includes LED's inside said Placido disk and said direction means comprises a beam splitter.

14. An integrated test pupil device for corneal topography for pointing out the actual pupil centre on the light alteration, comprising:
a videocamera in sequence with an optical group including a pair of lens, and an outside Placido disk;
a fixing LED and an infrared fixing LED provided with a neon glow lamp and a beam splitter plate; and
at least one Placido disk infrared LED for providing grazing light for a pupil test.

15. The device as claimed in claim 4, wherein said lateral LED and said fixing LED are infrared thereby not to interfere with light sources used for a cornea test that uses frequencies in the visible light field.

16. The device as claimed in claim 14, wherein said Placido disk infrared LED is placed inside said housing.

17. The device as claimed in claim 14, including a set of Placido disk infrared LED's in a parallel position relative to said neon glow lamp.

18. The device as claimed in claim 14, wherein only one of said Placido disk infrared LED is provided with a beam splitter plate and is in parallel with said neon glow lamp.

19. The device as claimed in claim 14, wherein the pupil centre to the light variation is filmed by said camera contemporaneously to provide a fixing centre and a pupil centre.

20. The device as claimed in claim 14, comprising a composite arrangement including said Placido disk, an infrared LED relative to and inside said Placido disk proximate to said beam splitter.
